(19) United States
(12) Patent Application Publication
(10) Pub. No.: US 2010/0076534 A1
(43) Pub. Date: Mar. 25, 2010

(54) MALLEABLE NEEDLE HAVING A PLURALITY OF ELECTRODES FOR FACILITATING IMPLANTATION OF STIMULATION LEAD AND METHOD OF IMPLANTING AN ELECTRICAL STIMULATION LEAD

(76) Inventor: William Alan Mock, Frisco, TX (US)

Correspondence Address: ST. JUDE MEDICAL NEUROMODULATION DIVISION, 6901 PRESTON ROAD, PLANO, TX, 75024, US

(21) Appl. No.: 11/877,256

(22) Filed: Oct. 23, 2007

Related U.S. Application Data
(60) Provisional application No. 60/862,899, filed on Oct. 25, 2006.

(51) Int. Cl. A61N 1/05

(52) U.S. Cl. 607/116

(57) ABSTRACT

In one embodiment, a malleable needle is provided at least three independent electrodes to facilitate the implantation of an electrical stimulation lead for peripheral nerve stimulation. The malleable characteristic of the needle enables the needle to be bent or shaped according to the patient anatomy. Once the needle is appropriately shaped by the physician, the physician inserts the needle into a prospective site for stimulation. The provision of the electrodes enables a suitable number of electrode patterns to be tested to determine whether the stimulation site is satisfactory. By utilizing the malleable needle in this manner, a number of stimulation sites can be tested in an efficient manner to identify an optimal location for implantation of the stimulation lead.

### RELATED APPLICATIONS

This application claims the benefit of U.S. Provisional Application No. 60/862,899, filed Oct. 25, 2006, the disclosure of which is fully incorporated herein.

### BACKGROUND

This application is generally related to a method of implanting a lead for peripheral nerve stimulation and peripheral nerve field stimulation using a malleable needle having multiple electrodes.

Peripheral nerves are nerves in the body other than the nerves of the brain or spinal cord. Peripheral nerve injury may result in the development of chronic intractable pain which in some patients may prove unresponsive to conservative pain management techniques. Peripheral Nerve Stimulation (PNS) has developed as a successful therapy for pain management when the pain is known to result from a specific nerve. Sweet and Wespic first used electrical stimulation of peripheral nerves in the 1960s to mask the sensation of pain with a tingling sensation (paresthesia) caused by the electrical stimulation. Subsequent refinements in the technology, surgical technique and patient selection have led to improved long term results.

Efforts have also been made to treat psychiatric disorders with peripheral/cranial nerve stimulation. For example, partial benefits with vagus nerve stimulation in patients with depression have been described in U.S. Pat. No. 5,299,569. U.S. Pat. No. 5,263,480 describes that stimulation of the vagus nerve may control depression and compulsive eating disorders. U.S. Pat. No. 5,540,734 teaches stimulation of the trigeminal or glossopharyngeal nerves for psychiatric illness, such as depression.

Another example of peripheral nerve stimulation includes stimulating the C2 dermatome area to treat occipital neuralgia, which may be defined generally as an intractable headache originating in the back of the head in the vicinity of the C2 dermatome area. This method of delivering electrical stimulation energy to the C2 dermatome area to treat occipital neuralgia involves positioning stimulation electrodes of an implantable electrical stimulation lead with at least one electrode in the fascia superior to in a subcutaneous region proximate the C2 dermatome area.

More recently, it has been reported that electrical stimulation by a lead implanted in the occipital region is useful to treat a number of disorders. U.S. Patent Application Publication No. 2006/0047325 by Thimineur et al. discloses that patients receiving electrical stimulation of the occipital region have exhibited surprisingly significant psychological and cognitive improvements. Also, U.S. Patent Application Publication No. 2006/0047325 has further disclosed that stimulation in this region has been shown to have an impact on Rheumatoid Arthritis, movement disorders, and obesity in patients.

The implantation of a stimulation lead within a patient typically occurs through a hollow needle. Specifically, the needle with a stylet inserted within the lumen of the needle is inserted within the patient to a location adjacent to the stimulation site. The stylet is removed and the stimulation lead is inserted through the lumen of the needle to the stimulation site. The needle is then slightly retracted to expose the electrodes of the stimulation lead to tissue. Electrical pulses can then be applied to the stimulation site through the stimulation lead to determine if the correct location has been reached. If the location is incorrect, the stimulation lead and needle are removed and the process is repeated.

It is also known to utilize a needle with an active tip to verify a stimulation location before lead implantation is attempted. For example, U.S. Pat. No. 6,971,393 to Mamo discloses inserting a needle with an active tip into a stimulation site adjacent to a sacral nerve root to test the effect of stimulation on the patient. Assuming that the test is successful, a larger needle is inserted in the patient to access the same location. A stylet is removed from the needle and a stimulation lead is inserted through the needle. Additional stimulation testing with the implanted lead can also occur if deemed appropriate by the physician.

### BRIEF SUMMARY

In one embodiment, a malleable needle is provided at least three independent electrodes to facilitate the implantation of an electrical stimulation lead for peripheral nerve stimulation. The malleable characteristic of the needle enables the needle to be bent or shaped according to the patient anatomy. Once the needle is appropriately shaped by the physician, the physician inserts the needle into a prospective site for stimulation. The provision of the electrodes enables a suitable number of electrode patterns to be tested to determine whether the stimulation site is satisfactory. By utilizing the malleable needle in this manner, a number of stimulation sites can be tested in an efficient manner to identify an optimal location for implantation of the stimulation lead.

The foregoing has outlined rather broadly various features and technical advantages in order that the detailed description that follows may be better understood. Additional features and/or advantages will be described hereinafter which form the subject of the claims. It should be appreciated that the conception and specific embodiment disclosed may be readily utilized as a basis for modifying or designing other structures for carrying out the same purposes. It should also be realized that such equivalent constructions do not depart from the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 depicts a cross-sectional view of a malleable needle for use in implanting a stimulation lead for peripheral nerve stimulation according to one representative embodiment.

FIG. 2 depicts a malleable needle having a plurality of electrodes for use in implanting a stimulation lead for peripheral nerve stimulation according to one representative embodiment.

FIG. 3 depicts a removable handle for the malleable needle shown in FIG. 2 according to one representative embodiment.

FIG. 4 depicts a trial stimulator coupled to a malleable needle for identifying a stimulation location for peripheral nerve stimulation according to one representative embodiment.

FIG. 5 depicts a cannula adapted to the malleable needle shown in FIG. 2 according to one representative embodiment.

FIG. 6 depicts a flowchart for implanting a stimulation lead according to one representative embodiment.

### DETAILED DESCRIPTION

Some representative embodiments provide a malleable needle having at least three independent electrodes that can be programmed in a tri-state manner to facilitate implantation of a stimulation lead for peripheral nerve stimulation. A physician may insert the needle into a prospective site for application of electrical pulses for the peripheral nerve stimulation or peripheral nerve field stimulation.

The needle is preferably malleable so that the physician can alter the needle's longitudinal shape. For example, if the peripheral nerve stimulation involves stimulating the occipital nerve area of a patient, the physician can shape the needle to conform to the general profile of the back of the patient's skull thereby allowing the needle to be maintained in or near the patient's fascia in the occipital region. Alternatively, the physician can alter the shape of the malleable needle to maneuver around an obstacle that impairs access to the desired peripheral nerve tissue.

Upon insertion into the respective site, the physician may couple the needle to a trial stimulator (i.e., a pulse generator). The physician can adjust the settings of the trial stimulator to test various stimulation pulse patterns. In particular, the physician can apply different electrode polarity combinations to the multiple electrodes in addition to varying pulse amplitude, pulse frequency, and pulse width. By applying the test stimulation pulses, the physician can determine whether the correct location has been selected and whether the stimulation will be effective for the patient's disorder.

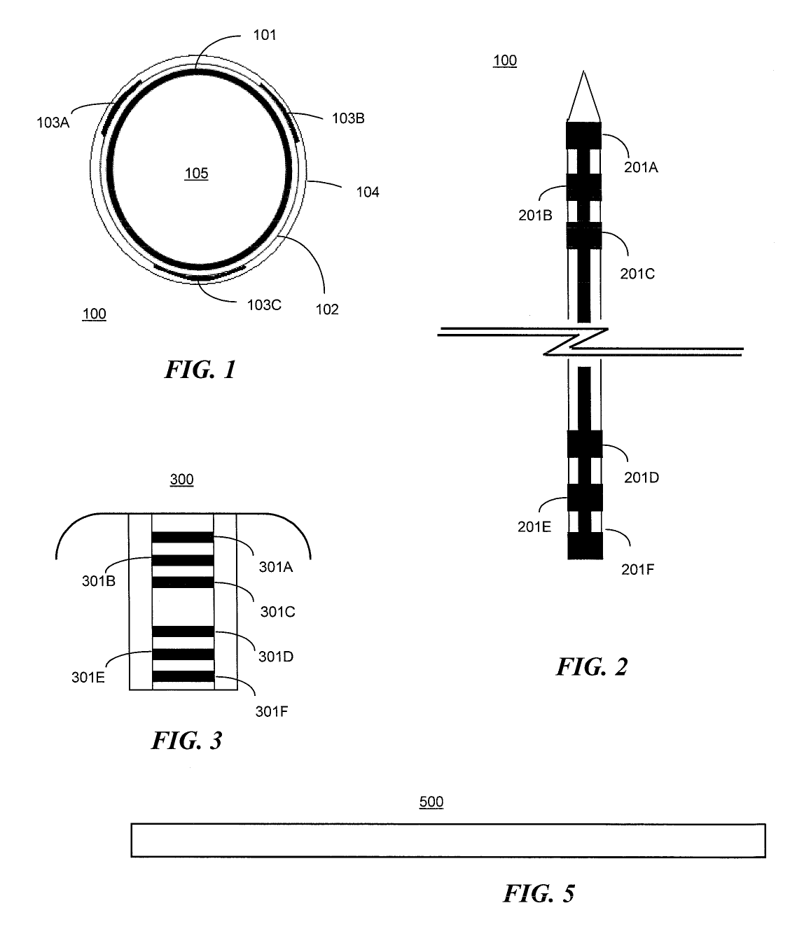

Referring now to the drawings, FIG. 1 depicts a cross-sectional view of malleable needle 100 for use in implanting a stimulation lead for peripheral nerve stimulation according to one representative embodiment. In one embodiment, needle 100 comprises core 101 of medical grade stainless steel. In another embodiment, core 101 can comprise any medical grade material that can be formed into a suitable needle as described below. The thickness of core 101 is preferably selected to allow needle 100 to be bent or shape as desired by the physician. Needle 100 may also include deformable material 105 (such as silicone or other deformable material) within the lumen of core 101. The deformable material 105 preferably possesses a sufficiently low durometer that allows core 101 to be readily bent or shaped. Also, the selection of the durometer of deformable material 105 should permit distribution of the applied bending/shaping force to prevent core 101 from being collapsed at a particular point or developing an undesired crimp. In another embodiment, a stylet such as is described in U.S. Published application Ser. No. 10/637,342, which is incorporated by reference herein, may be used with the present invention.

To provide electrical isolation between the various electrical contacts 201, core 101 is coated with insulative material 102 such as a dielectric epoxy material. A surfactant can be added to insulative material 102 to facilitate the coating of core 101. Additionally or alternatively, a vacuum deposit process can be used to apply a crystal or glass dielectric material on core 101. Once, the insulative layer 102 is applied, independent electrical traces 103 are applied around the circumference of the needle, e.g., by vacuum deposition. In alternative embodiment, a solid layer of conductive material, such as MP35N or other suitable conductor, could be deposited on the outer surface of core 101 and a suitable YAG or other laser could be utilized to etch trenches in the conductive material to define independent traces 103. The etching of trenches causes each trace 103 to be electrically isolated from the other traces 103. As shown in FIG. 1, traces 103 are disposed as discrete conductive paths that run longitudinally along needle 100. Also, three traces 103A-103C are applied to support three independent electrodes 201, although more traces 103 could be provided to support additional electrodes for alternative embodiments. After the traces 103 are provided, another insulative layer 104 is deposited to maintain the electrical isolation of traces 103 from each other.

FIG. 2 depicts a longitudinal view of malleable needle 100 according to one representative embodiments. As shown in FIG. 2, needle 100 comprises three electrical contacts 201A-201C at a distal end of the needle to function as electrodes. Likewise, needle 100 comprises three electrical contacts 201D-201F at a proximal end of needle 100. Each contact 201 is preferably implemented in a similar manner. Specifically, a portion of insulative layer 104 is removed above a respective trace 103 wherein the contact 201 is to be formed. Then, conductive material is applied or deposited in a ring-like manner around the needle and over the exposed portion of the respective trace 103. Accordingly, the ring is then electrically coupled to the exposed trace 103 and is also electrically isolated from the other traces 103. After the contacts 201 are formed, each trace 103 is coupled to two contacts 201, i.e., one contact 201 at the proximal end and one contact 201 at the distal end. Thus, application of electrical energy at the contact 201 at the proximal end is conducted by the respective trace 103 to the contact 201 at the distal end.

FIG. 3 depicts removable handle 300 for malleable needle 100 according to one representative embodiment. Handle 300 preferably provides a convenient structure to permit the physician to manipulate needle 100 through the tissue of the patient in an efficient manner. Also, handle 300 preferably provides an integral connector portion (electrical contacts 301A-301F) to provide a temporary electrical connection between electrodes of a trial lead/cable with the electrodes 201A-201C of the needle during stimulation testing.

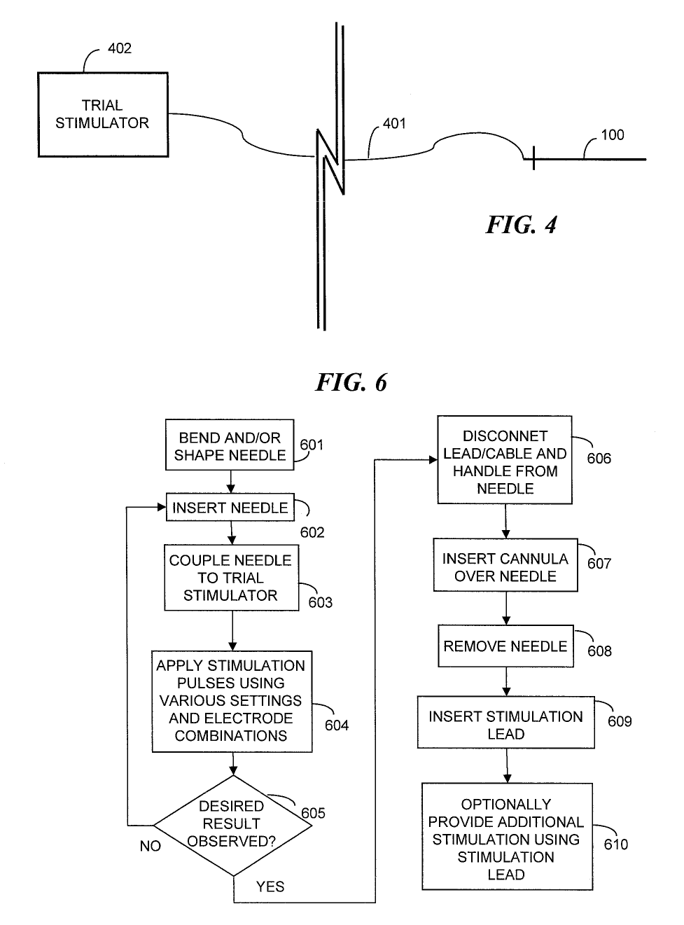

FIG. 4 depicts trial stimulator 402 coupled to malleable needle 100 through stimulation lead 401 for identifying a stimulation location for peripheral nerve stimulation according to one representative embodiment. The physician uses stimulator 402 to generate the electrical pulses to stimulate the nerve tissue of the patient. The stimulator 402 allows the physician to control the amplitude, frequency, and pulse width of the stimulation pulses. Also, the stimulator 402 allows the physician to control the electrode polarities (i.e., each electrode of needle 100 can function as an anode or cathode or function in a high-impedance state). If the physician is unable to obtain satisfactory results from the stimulation after trying a number of different stimulation settings and electrode patterns, the needle is most likely not positioned sufficiently close to the respective nerve tissue. The physician can then readily remove the needle 100 from the current location and replace the needle in another location.

Handle 300 as shown in FIG. 3 is preferably readily removable from needle 100 to permit a cannula to be inserted over needle 100 and through the tissue of the patient when the correction stimulation location has been identified. FIG. 5 depicts cannula 500 adapted to malleable needle 100 according to one representative embodiment. Cannula 500 preferably possesses an inner diameter that is slightly greater than the outer diameter of needle 100 and the stimulation lead to be implanted. Cannula 500 can be constructed of any suitable biocompatible material such as stainless steel or various higher durometer polymers. After cannula 500 is inserted over the needle to the stimulation location, needle 100 is removed to allow the stimulation lead to be inserted through cannula 500 to the stimulation site.

Although some implementation details have been discussed in regard to FIGS. 1-4, other implementations of a malleable needle having a plurality of electrodes can be employed according to alternative embodiments. For example, in lieu of providing electrical traces along the exterior of core 101, some embodiments may provide conductive wires within the core 101. For example, a stimulation lead of insulative material embedding conductive wires can be provided within the lumen of core 101. Apertures can be provided through core 101 to access the stimulation lead, e.g., by laser ablation. A respective aperture can also be provided through a portion of the insulative material of the stimulation lead to access a respective conductive wire. Conductive material can be deposited or otherwise built-up to provide a conductive path to the exterior where an electrical contact is to be formed. Alternatively, a linking wire or bridge can be coupled to the exposed conductive wire of the interior stimulation lead.

FIG. 6 depicts a flowchart for implanting a stimulation lead according to one representative embodiment.

In step 601, needle 100 is bent and/or otherwise shaped to conform to the anatomy of a patient near the location where a lead is to be implanted for peripheral nerve stimulation. Alternatively, the needle can be bent and/or otherwise shaped to allow the physician to avoid any obstructions between the insertion point of the needle and the desired stimulation location.

In step 602, the needle 100 is inserted into the patient tissue and, in step 603, the needle is coupled to a trial stimulator through a suitable lead or cable. In step 604, stimulation pulses are delivered from the trial stimulator through the needle 100 according to a number of different stimulation settings. The electrode polarities can be varied as well as the pulse amplitude, pulse frequency, and pulse width.

In step 605, a decision is made whether the desired result of the peripheral nerve stimulation has been observed (e.g., relief from pain, change in a psychological state, change in cognitive functioning, change in one or several physiological functions, etc.). If the desired result has not been achieved (and the physician has not concluded that it cannot be achieved), the needle is withdrawn and the process returns to step 602 in an attempt to identify another more appropriate stimulation location.

If the desired result of the stimulation has been observed, the connecting electrical lead/cable is removed and also the handle of the needle is removed (step 606). In step 607, a cannula is inserted over the needle to the stimulation site. In step 608, the needle is removed from the cannula. In step 609, a stimulation lead is inserted through the cannula to the stimulation site. In step 610, further stimulation can be optionally provided through the stimulation lead to verify the correct placement of the stimulation lead.

Although some representative embodiments and advantages have been described in detail, it should be understood that various changes, substitutions and alterations can be made herein without departing from the appended claims. Moreover, the scope of the appended claims is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed:

1. A malleable needle for facilitating implantation of a stimulation lead for peripheral stimulation, comprising:
a longitudinally extending flexible core capable of being shaped by a physician to a patient's anatomy;
at least three electrodes disposed at a distal end of the malleable needle;
at least three electrical conductors each coupled to a respective one of the at least three electrodes and extending longitudinally along the needle; and
a connector structure for making temporary electrical contact with a stimulation lead or cable for conducting stimulation pulses from the stimulation lead or cable to the at least three electrodes.

2. The malleable needle of claim 1 wherein the at least three electrical conductors are conductive traces disposed exterior to the core and between respective insulative layers.

3. The malleable needle of claim 1 wherein the at least three electrical conductors are conductive wires embedded in an insulative body.

4. The malleable needle of claim 3 wherein the insulative body that embeds the at least three electrical conductors is disposed in a lumen of the core.

5. The malleable needle of claim 1 wherein the connector structure is embedded within a handle structure.

6. The malleable needle of claim 5 wherein the handle structure is removable from the core.

7. The malleable needle of claim 1 wherein the core is fabricated from stainless steel.

8. The malleable needle of claim 1 wherein a deformable material is disposed in an interior of the core to prevent crimping of the core when the core is shaped by a physician.

9. A method of implanting a stimulation lead within a patient for peripheral nerve stimulation, the method comprising:
inserting a malleable needle into a prospective stimulation site proximate to a peripheral nerve, wherein the malleable needle comprises at least three electrodes;
applying stimulation pulses to the prospective stimulation site using the at least three electrodes;
when an expected result of the peripheral nerve stimulation is observed, inserting a cannula over the malleable needle into patient tissue near the prospective stimulation site;
after inserting the cannula, removing the malleable needle from the cannula; and
inserting a stimulation lead through the cannula such that electrodes of the stimulation lead are disposed proximate to the prospective stimulation site.

10. The method of claim 9 further comprising:
shaping the malleable needle to conform to patient anatomy.

11. The method of claim 10 wherein the malleable needle is shaped to conform to an occipital region of the patient.

12. The method of claim 9 further comprising:
adjusting the shape of the malleable needle to avoid an obstruction in the patient's tissue that blocks access to the prospective stimulation site.

13. The method of claim 9 further comprising:
when an expected result of the peripheral nerve stimulation is not initially observed, (i) removing the needle; (ii) selecting another prospective stimulation site; and (iii) repeating application of stimulation pulses using the at least three electrodes of the malleable needle at the another prospective stimulation site.

14. The method of claim 9 further comprising:
removing a handle structure from the malleable needle to permit insertion of the cannula over the malleable needle.

15. The method of claim 9 wherein the malleable needle comprises conductive traces disposed exterior to a core of the malleable needle and between respective insulative layers to provide conductive paths to the at least three electrodes.

16. The method of claim 9 wherein the malleable needle comprises conductive wires within an interior of the malleable needle to provide conductive paths to the at least three electrodes.

17. The method of claim 16 wherein the conductive wires are embedded in an insulative body.

18. The method of claim 9 further comprising:
removing a removable handle structure from the needle.

19. The method of claim 18 wherein the removable handle comprises a connector structure for electrically connecting an electrical cable or lead from a trial stimulator to the at least three electrodes of the malleable needle.

20. The method of claim 9 wherein a deformable material is disposed in a lumen of the malleable needle to prevent crimping of the core when the core is shaped by a physician.
